(19) United States
(12) Patent Application Publication
(10) Pub. No.: US 2021/0308315 A1
(43) Pub. Date: Oct. 7, 2021

(54) Fragrance Fan

(71) Applicant: O2COOL, LLC, Chicago, IL (US)

(72) Inventors: Tom Zeng, Shenzhen (CN); Eric F. Junkel, Mount Prospect, IL (US)

(21) Appl. No.: 16/839,545

(22) Filed: Apr. 3, 2020

(51) Int. Cl. A61L 9/12; F04D 25/10

(52) U.S. Cl. CPC A61L 9/122; A61L 2209/133; F04D 25/10

(57) ABSTRACT

A fragrance fan includes a housing, a grille, a fan, a motor, and a power supply. The grille includes integrally formed fragrance medium receptacles for removably receiving and supporting fragrance medium members in an airstream produced when the motor rotates the fan about a fan axis.

### BACKGROUND

This disclosure pertains to fragrance dispersing fans.

### SUMMARY

An aspect of the present disclosure is a fragrance fan comprising a housing, a motor, at least one grille, and a fan. The grille is mounted to the housing and comprises at least one fragrance medium receptacle integrally formed therewith. The fragrance medium receptacle is adapted and configured to retain a fragrance medium member. The is fan rotatably disposed in the housing about a fan axis and operatively connected to the motor. The fan is adapted and configured to be driven by the motor to rotate about the fan axis when the motor is energized, and when driven by the motor to rotate about the fan axis, to force air through the grille incident to the fragrance medium receptacle and to force air from an interior of the housing to an exterior of the housing.

Further features and advantages, as well as the operation, are described in detail below with reference to the accompanying drawings.

### BRIEF DESCRIPTION OF THE DRAWINGS

FIG. 1 is a bottom rear right perspective view of a fan in accordance with the disclosure.

FIG. 2 is a right side elevation view of the fan of FIG. 1.

FIG. 3 is a left side elevation view of the fan of FIG. 1.

FIG. 4 is a front elevation view of the fan of FIG. 1.

FIG. 5 is a rear elevation view of the fan of FIG. 1.

FIG. 6 is a top plan view of the fan of FIG. 1.

FIG. 7 is a bottom plan view of the fan of FIG. 1.

FIG. 8 is a top front left perspective view of the fan of FIG. 1.

FIG. 9 is a partial, enlarged, rear perspective view of the fan of FIG. 1.

FIG. 10 is a partial, enlarged, rear elevation view of fan of FIG. 1.

FIG. 11 is a partial, enlarged front elevation view of the fan of FIG. 1.

Reference numerals in the written specification and in the figures indicate corresponding items.

### DETAILED DESCRIPTION

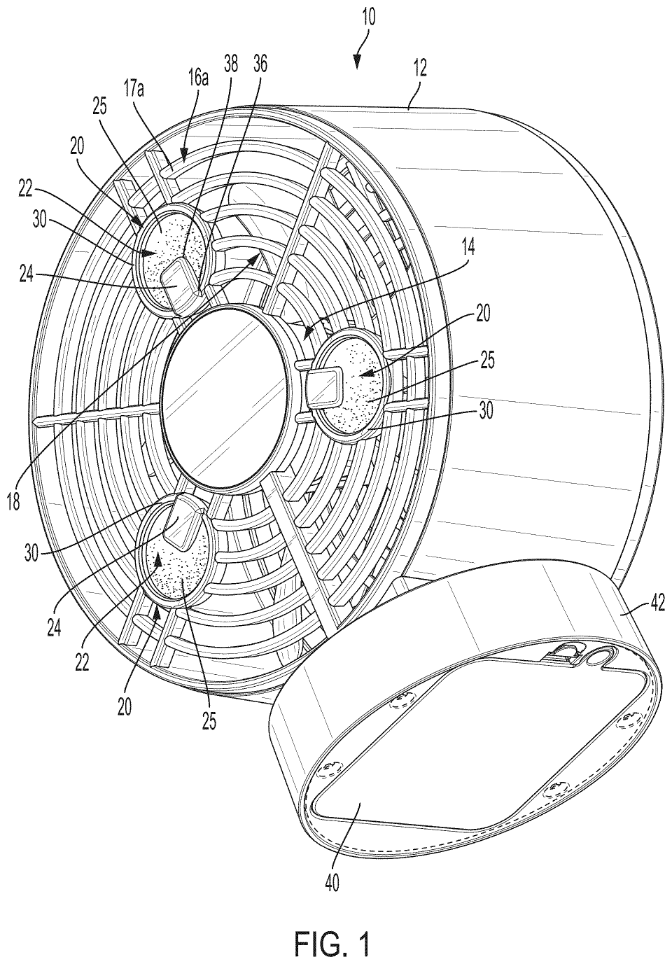

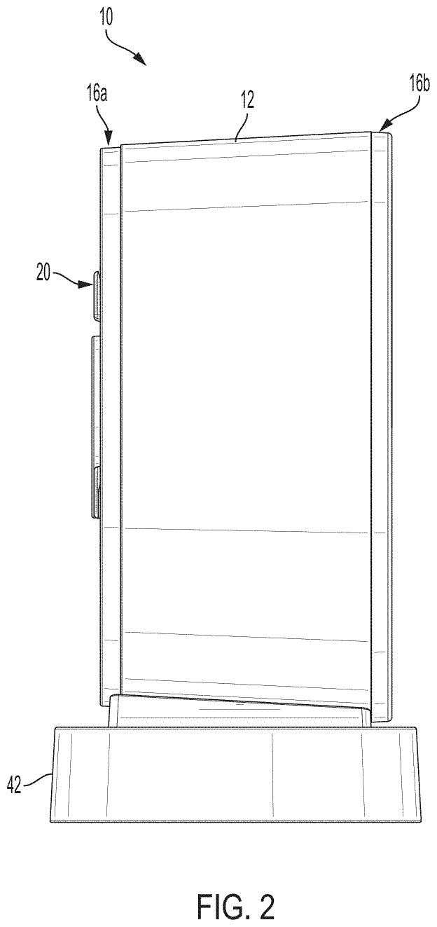

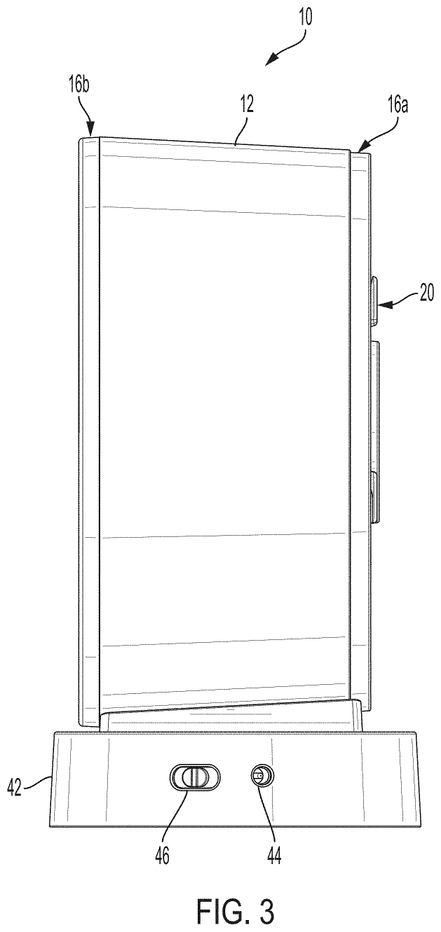

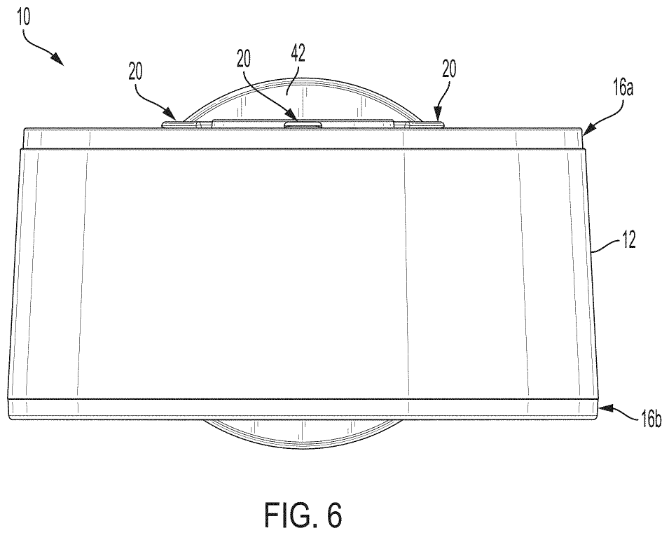

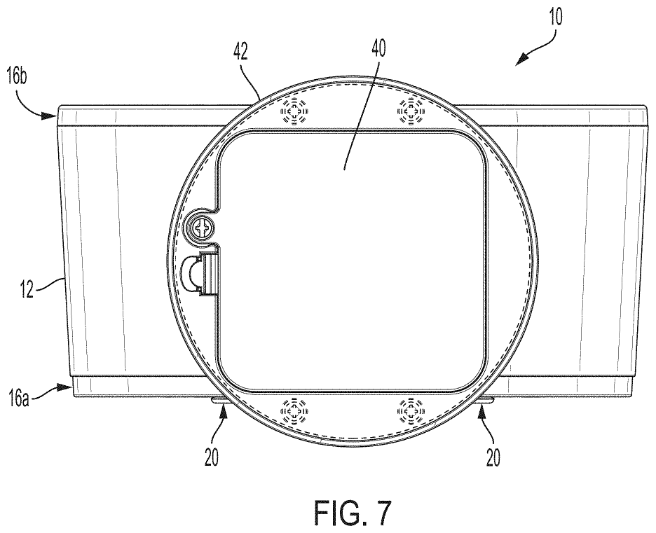

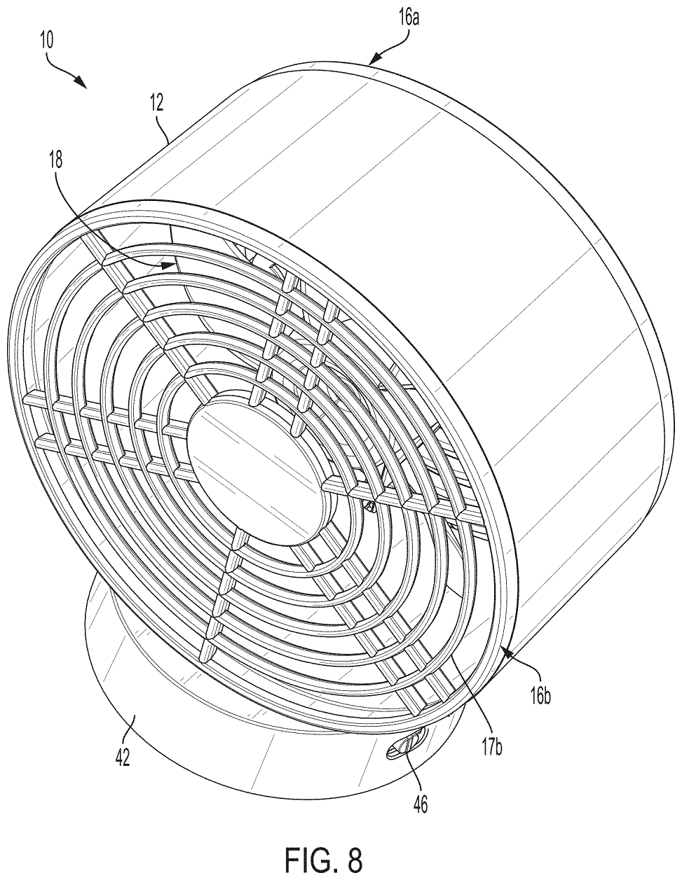

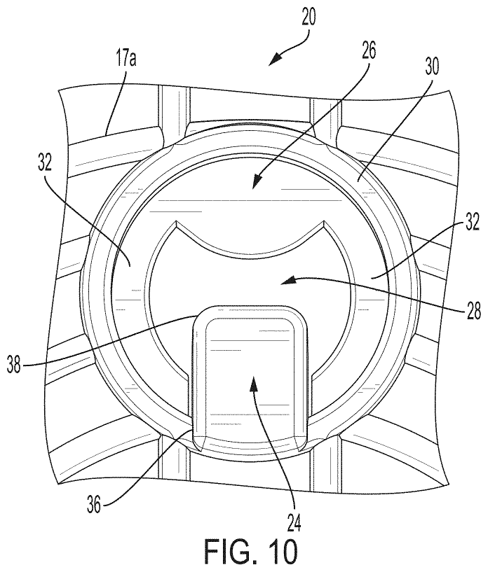

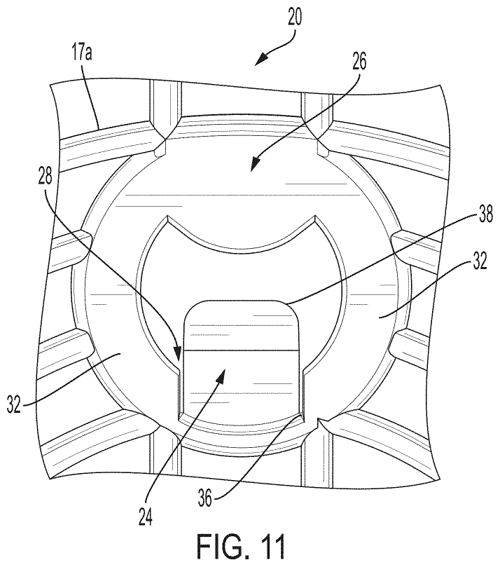

An embodiment of a fragrance fan 10 in accordance with the present disclosure is illustrated in FIGS. 1-11. The fragrance fan 10 comprises a housing 12, a motor 14 (indicated in FIG. 1), and at least one grille 16a,16b, mounted to the housing 12. The housing 12 may be generally cylindrical or may taper inwardly or outwardly from an air intake side to an air discharge side (e.g., may be generally conical). An outer diameter of housing 12 may be approximately four to six inches, so that the fragrance fan 10 may be conveniently stored and/or used on a desk, tabletop, or shelf. For purposes of illustration making reference to the drawings, an intake grille 16a is mounted to the rear of the housing 12, and an outlet grille 16b is mounted to the front of the housing. While two grilles 16a,16b are shown in the drawings, only one may be provided. The at least one grille 16a,16b may comprise a plurality of grille ribs 17a,17b. The grille construction of the illustrated embodiment has circumferential ribs that are relatively closely spaced and radial ribs that are spaced farther from one another. Other grille constructions, not shown, may instead include more closely spaced radial ribs and/or circumferential ribs that are spaced farther from one another. For example, other grille constructions may simply include a larger number of radial ribs extending outwardly from an inner circumference of the grille. Still other grille constructions may include a first set of radial ribs originating and extending outwardly from the grille inner circumference and one or more subsequent sets of radial ribs originating and extending outwardly from an intermediate circumferential rib, to divide the expanding circumferential gaps between adjacent ribs of the first set.

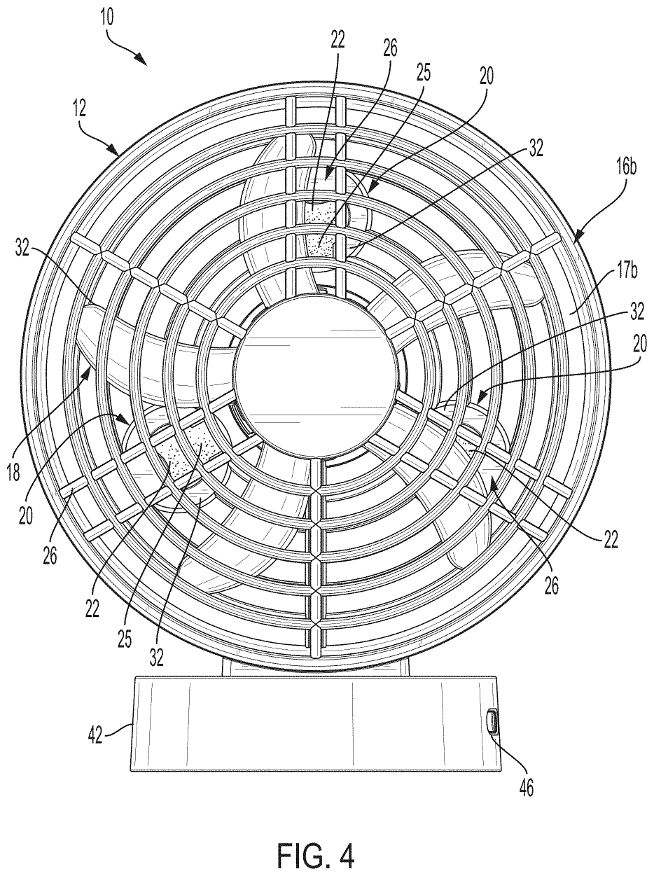

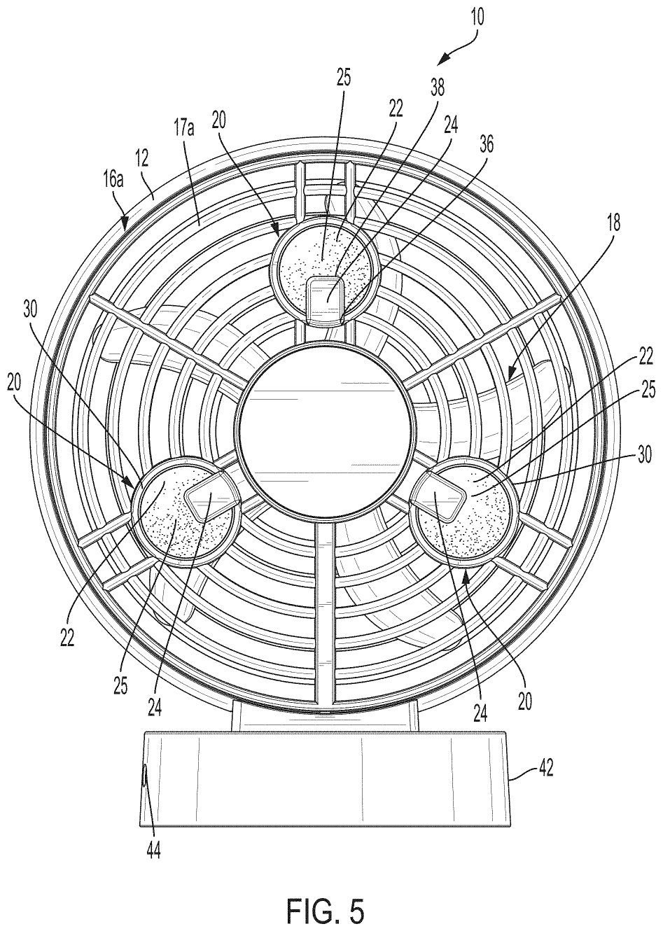

The fragrance fan includes a fan 18 rotatably disposed in the housing 12 about a fan axis. The motor 14 may be operatively connected to the fan 18 and configured, when energized, to drive rotation of the fan 18 about the fan axis. In the illustrated embodiment, the fan 18, as best seen in FIGS. 4 and 5, is an axial flow fan, adapted and configured, when driven by the motor 14 to rotate about the fan axis, to generate an axial forced airflow, and in particular, to force (draw) air axially through the intake grille 16a incident to the fragrance medium receptacle and to force (impel) the air axially from an interior of the housing 12 to an exterior of the housing 12 through the outlet grille 16b. It will be appreciated that the present disclosure may alternatively be embodied in a device using a centrifugal discharge fan, adapted and configured to generate a forced airflow that is generally axial at an intake grille and generally orthogonal to the fan axis at an outlet grille. The terms “upstream” and “downstream” may be used to describe structures in relation to a forced airflow direction, regardless of fan type.

The grille according to the present disclosure includes at least one fragrance medium receptacle formed integrally therewith. In the illustrated embodiment, the intake grille 16a includes three fragrance medium receptacles 20, and the grille 16a, grille ribs 17a, and the fragrance medium receptacles 20 may be formed integrally and/or monolithically. The fragrance medium receptacle 20 is adapted and configured to retain a fragrance medium member 22, as shown in FIGS. 1, 4, and 5. The fragrance medium receptacles 20 may be equiangularly spaced apart from one another circumferentially about the fan axis. Alternatively, the intake grille may comprise more than three or fewer than three fragrance medium receptacles integrally formed therewith. The fragrance medium receptacles may be the same size or have different sizes. Larger fragrance medium receptacles, and/or a greater number of fragrance medium receptacles, may be desired in the case of a fan having a larger fan grille. The fragrance medium receptacles may also be provided on the outlet grille.

The fragrance medium receptacle 20 may include a clip 24 generally in a plane perpendicular or oblique to the fan axis. The clip 24 may be adapted and configured to removably secure a fragrance medium member 22 in the fragrance medium receptacle 20. In an embodiment, the clip 24 is relatively rigid and immovable relative to the intake grille 16a, as may be desired when the fragrance medium member 22 is itself a flexible and/or compressible member. In the illustrated embodiment, the fragrance medium member 22 is a generally planar (i.e., broad and thin; having a body lying close to a plane) and flexible member. The fragrance medium member 22 has opposite faces 25, one of which faces rearward when the fragrance medium member 22 is received in the fragrance medium receptacle 20, as shown in FIGS. 1 and 5, and the other of which faces forward, as shown in FIG. 4. The fragrance medium member faces 25 may generally have a common diameter and be spaced from each other by a generally uniform thickness. The fragrance medium member 22 may, for example, be a commonly available 22-mm diameter felt round, having a generally uniform thickness of about 3 mm.

In an embodiment, the grille 16a and the fragrance medium receptacle 20 (inclusive of the clip 24) are monolithically formed. For example, the grille 16a along with grille ribs 17a may be a single piece of a molded plastic. The material or materials of the grille 16a may be chemically resistant to fragrance chemicals, such as aroma compounds and substances used in fragrances as carriers for aroma compounds. Suitable chemically resistant resins for a molded grille may include polypropylene and polyethylene resins. The fragrance medium receptacle 20 can provide advantages over existing air freshener products that include a separate housing or body designed to attach to a fan grille. For example, the integrally and/or monolithically formed fragrance medium receptacle can provide improved durability, reduced airflow obstruction, and increased exposure of a fragrance medium to airflow.

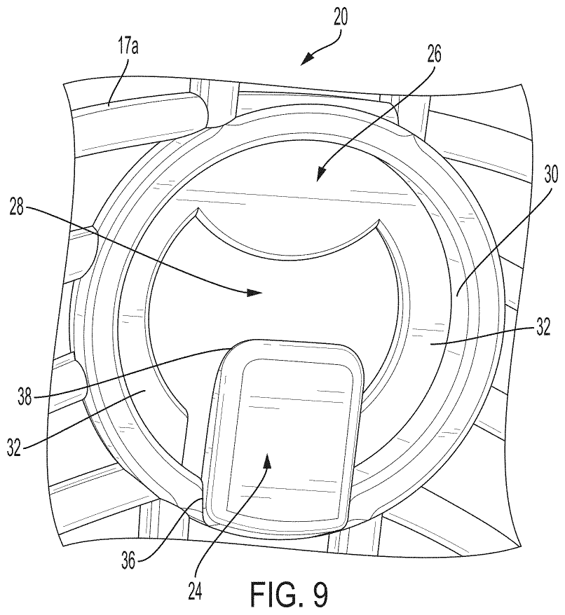

The fragrance medium receptacle 20 is shown holding the fragrance medium member 20 in FIGS. 1, 4, and 5. In FIGS. 9-11, the fragrance medium receptacle 20 is shown separately in greater detail, without the fragrance medium member 22. The fragrance medium receptacle 20 may have a receptacle panel 26 generally in a plane. The plane of the receptacle panel is perpendicular to the forced airflow direction (i.e., in the illustrated embodiment, perpendicular to the fan axis), and the receptacle panel 26 is spaced downstream of the clip 24, toward an interior of the housing 12. In other embodiments, in which a receptacle is integrally formed with an outlet grille, a receptacle panel may be spaced upstream of a receptacle clip. The clip 24 may be disposed opposite and spaced from the panel 26 to form a slot 28 generally in a plane parallel to the plane of the panel 26, and thus facing generally in the forced airflow direction. The slot 28 may be adapted and configured to removably receive the fragrance medium member 22. The fragrance medium receptacle 20 may have an outer periphery comprising a rim 30, which may be a continuous rim. The rim 30 may generally circumscribe a perimeter of the receptacle adjacent to the slot 28. When a fragrance medium member 22 is received in the slot 28, the rim 30 is adapted and configured to support the fragrance medium member 22 and restrain the fragrance medium member from moving in the plane of the slot away from a position in which it is received in the slot.

The fragrance medium receptacle 20 is preferably adapted and configured to retain a generally planar, flexible fragrance medium member such as the fragrance medium member 22. For example, the slot 28 itself may be generally planar, and the clip 24 substantially rigid. The clip 24 may be disposed opposite and spaced from the panel 26 in a manner such that a fragrance medium member 22 inserted in the fragrance medium receptacle 20 is removably secured against the panel 26 by the clip 24. For example, the slot 28 may have a depth dimension extending from the clip 24 to the receptacle panel 26, which is approximately equal to or less than a relaxed thickness of the flexible and compressible fragrance medium member 22. Thus, the fragrance medium member may be slightly compressed when inserted into the receptacle. To accommodate a fragrance medium member that is instead a solid disk, the depth dimension of the slot 28 may be approximately equal to a thickness of the solid disk, and the clip 24 may be flexible and/or pivotally connected to the grille 16a to permit the solid disk to snap into place in the slot 28 once the fragrance medium member is inserted into the receptacle.

The receptacle panel 26 may comprise two prongs 32, and the clip 24 may comprise a third prong, which collectively provide a staggered-fork configuration for the receptacle. The clip 24 may have a clip proximal end 36 connected to the rim 30 of the fragrance medium receptacle 20 and a clip distal end 38 extending away from the clip proximal end 36. In the illustrated embodiment, the clip proximal end 36 is radially inward of the clip distal end 38, with respect to the fan axis. In other axial-flow fan embodiments, the clip attachment to the receptacle may be reversed with the clip proximal end radially outward of the clip distal end with respect to the fan axis. The prongs 32 may define an open area on a front side of the slot 28 extending therebetween. The slot front side open area may overlie at least a portion of the clip 24 in a plane parallel to that of the panel 26, and more preferably eclipse substantially all of the clip 24 in the plane parallel to that of the panel 26. Likewise, an open area on a rear side of the slot 28 may generally surround a portion of the clip 24 that extends distally from the clip proximal end 36. The slot rear side open area may overlie at least a portion of the receptacle panel 26, and more preferably eclipse substantially all of the panel 26 in the plane parallel to that of the panel 26. In this configuration, the prongs 32 and the clip 24 hold the fragrance medium member 22 in such a manner that, at substantially all locations on the fragrance medium member 22 in the plane parallel to that of the panel 26, at least one of the opposite faces 25 of the fragrance medium member 22 is exposed to air.

The fragrance medium member 22 may be formed of a generally solid material, which may be porous, thereby permitting airflow produced by the fan 18 to penetrate into and/or through the porous medium. A suitable fragrance medium may, for example, be a flexible, compressible, absorbent fabric such as felt, as in the felt round mentioned above. The fragrance medium may instead be a rigid body, such as a solid disk, and/or the fragrance medium member may comprise a fragrance medium held by a rigid support or housing (not shown), the support or housing preferably including openings (e.g. perforations) to permit airflow to reach the fragrance medium. The fragrance, which may be in a liquid (e.g., oil) or viscous solid (e.g., wax) form, contains at least one aroma compound, which is absorbed, adsorbed, or otherwise held in the fragrance medium.

Preferably, the fragrance fan 10 further comprises an internal power supply 40 operatively connected to the motor 14 to supply power to the motor 14. In the illustrated embodiment, the fragrance fan 10 includes a base 42 connected below the housing 12, and the internal power supply 40 is housed in the base 42. The internal power supply 40 may comprise one or more batteries, which may be of a standard removable and/or disposable type, such as AA or AAA batteries. A power switch 46 may be mounted to the base 42 for switching power on and off to the motor. The power switch 46 may have three positions to provide two motor/fan speeds and an off state. In addition to the internal power supply 40, the fragrance fan 10 may have a low-voltage DC power port 44 for connecting an external DC power supply (not shown). Alternatively, the internal power supply may comprise an integrated battery, such as a rechargeable lithium ion battery, which may be recharged through a charging port (not shown) on an outer surface of the base. The base 42 is adapted and configured to support the housing 12 while the base 42 rests on a flat horizontal surface. An external power supply may also be provided.

It should also be understood that when introducing elements of the present invention in the claims or in the above description of exemplary embodiments of the invention, the terms “comprising,” “including,” and “having” are intended to be open-ended and mean that there may be additional elements other than the listed elements. Additionally, the term “portion” should be construed as meaning some or all of the item or element that it qualifies. Moreover, use of identifiers such as first, second, and third should not be construed in a manner imposing any relative position or time sequence between limitations.

As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. A fragrance fan comprising
a housing;
a motor;
at least one grille mounted to the housing, the grille comprising at least one fragrance medium receptacle integrally formed therewith, the fragrance medium receptacle being adapted and configured to retain a fragrance medium member; and
a fan rotatably disposed in the housing about a fan axis, the fan being operatively connected to the motor, the fan being adapted and configured to be driven by the motor to rotate about the fan axis when the motor is energized, and when driven by the motor to rotate about the fan axis, to force air through the grille incident to the fragrance medium receptacle and to force air from an interior of the housing to an exterior of the housing.

2. The fragrance fan of claim 1, wherein the fragrance medium receptacle includes a clip adapted and configured to removably secure a fragrance medium member in the fragrance medium receptacle.

3. The fragrance fan of claim 2, wherein the grille and the fragrance medium receptacle are monolithically formed.

4. The fragrance fan of claim 1, wherein the at least one grille comprises an intake grille and an outlet grille, and the at least one fragrance medium receptacle is disposed on the intake grille.

5. The fragrance fan of claim 1, wherein the fragrance medium receptacle is adapted and configured to retain a generally planar shaped fragrance medium member.

6. The fragrance fan of claim 1, wherein the fragrance medium receptacle has a receptacle panel generally in a plane, the fan being adapted and configured, when driven by the motor to rotate about the fan axis, to force air through the grille in a direction generally facing the plane of the receptacle panel.

7. The fragrance fan of claim 6, wherein the clip is opposite and spaced from the panel to form a slot, the slot being adapted and configured to removably receive a fragrance medium member.

8. The fragrance fan of claim 6, wherein the clip is opposite and spaced from the panel in a manner such that a fragrance medium member inserted in the fragrance medium receptacle is removably secured against the panel by the clip.

9. The fragrance fan of claim 6 wherein the panel comprises an opening that overlies at least a portion of the clip in a plane parallel to the plane of the receptacle panel.

10. The fragrance fan of claim 2 wherein the clip has a proximal end connected to the fragrance medium receptacle and a distal end extending away from the proximal end.

11. The fragrance fan of claim 11 wherein the clip proximal end is radially inward of the clip distal end.

12. The fragrance fan of claim 1 wherein the fragrance medium receptacle has an outer periphery with a rim adapted and configured to support a fragrance medium member inserted in the fragrance medium receptacle.

13. The fragrance fan of claim 1, further comprising a fragrance medium member disposed in the fragrance medium receptacle.

14. The fragrance fan of claim 13, wherein the fragrance medium member comprises a fragrance medium and a fragrance stored in the fragrance medium.

15. The fragrance fan of claim 14 wherein the fragrance comprises a liquid absorbed into the fragrance medium.

16. The fragrance fan of claim 14, wherein the fragrance medium is felt.

17. The fragrance fan of claim 1, wherein the fan is an axial-flow fan.

18. The fragrance fan of claim 1, further comprising an internal power supply operatively connected to the motor to supply power to the motor.

19. The fragrance fan of claim 1, further comprising a base connected below the housing, the base being adapted and configured to support the housing and to rest on a flat horizontal surface.
